(19) United States
(12) Patent Application Publication
(10) Pub. No.: US 2015/0377891 A1
(43) Pub. Date: Dec. 31, 2015

(54) Methods for Predicting the Survival Time of Patients Suffering from Diffuse Large B-Cell Lymphomas

(71) Applicants: (INSTITUTE NATIONAL DE LA SANTÉ ET DE LA RECHERE MÉDICALE), Paris (FR); UNIVERSITE DE RENNES 1, Rennes (FR); ETABLISSEMENT FRANCAIS DU SANG, La Plaine Saint Denis Cedex (FR); CHU de Rennes, Rennes (FR)

(72) Inventor: Thierry FEST, Rennes Cedex (FR)

(21) Appl. No.: 14/766,510

(22) PCT Filed: Feb. 7, 2014
(86) PCT No.: PCT/EP2014/052449

§ 371 Date: Aug. 7, 2015

(30) Foreign Application Priority Data
Feb. 7, 2013 (EP) 13305145.8

(51) Int. Cl. G01N 33/574

(52) U.S. Cl. CPC G01N 33/57492; G01N 2333/705; G01N 2800/52

(57) ABSTRACT

The present invention relates to methods and kits for predicting the survival time of a patient suffering from a diffuse large B-cell lymphoma (DLBCL). In particular, the present invention relates to a method for predicting the survival time of a patient suffering from a diffuse large B-cell lymphoma (DLBCL) comprising the step of i) determining the level of sPD-L1 in a blood sample obtained from the patient ii) comparing the level determined at step i) with a predetermined reference value and iii) concluding that the patient has a poor prognosis when the level determined at step i) is higher than the predetermined reference value or concluding that the patient has a good prognosis when the level determined at step i) is lower than the predetermined reference value.

### FIELD OF THE INVENTION

The present invention relates to methods and kits for predicting the survival time of a patient suffering from a diffuse large B-cell lymphoma (DLBCL).

### BACKGROUND OF THE INVENTION

Diffuse large B-cell lymphoma (DLBCL) is the most common non-Hodgkin's lymphoma and is curable with anthracycline-based chemotherapy regimens associated to rituximab immunotherapy. The best available clinical tool to risk-stratify patients with diffuse large B-cell lymphoma at diagnosis is the International Prognosis Index (IPI); however, this scoring does not provide insight into the underlying tumor biology and molecular heterogeneity inherent to this disease¹.

Endogenous immune response to cancer may be counterbalanced by number of immune “checkpoints”, which normally terminate immune response after antigen activation, used by tumors to actively evade immune destruction^{2, 3}. Studies in animals have shown that inhibition of these checkpoints restore immune activation against cancer cells leading to new therapeutic avenues recently explored in advanced cancer³⁻⁶. Programmed death 1 (PD-1) is a key immune-checkpoint receptor expressed by activated T cells, and it mediates immunosuppression. Since in tumor tissues, activated T cells can encounter the immunosuppressive PD-1 ligands PD-L1 (B7-H1) and PD-L2 (B7-DC), both expressed by tumor cells and stromal cells, it was recently showed that the blockade of PD-1 or PD-L1 by monoclonal antibodies may lead to significant antitumor effects^{4, 6}. In diffuse large B-cell lymphoma the expression of PD-L1 by tumor cells has been reported and tumor associated T cells express PD-1^{7, 8}.

### SUMMARY OF THE INVENTION

The present invention relates to a method for predicting the survival time a patient suffering from a diffuse large B-cell lymphoma (DLBCL). The present invention also relates to methods of treating diffuse large B-cell lymphoma.

### DETAILED DESCRIPTION OF THE INVENTION

The dosage of soluble form of programmed death ligand 1 (sPD-L1) protein, one of the two ligands of programmed death 1 (PD-1) inhibitor receptor expressed by T cells, in the blood of adults with DLBCL has never been done. The inventors assessed the clinical interest of this dosage at the time of diagnosis for patients who underwent chemotherapy for aggressive form of diffuse large-B-cell lymphoma. They measured PD-L1 in the plasma of 288 patients that were enrolled in a multicenter, randomized trial that compared high-dose therapy associated with rituximab plus autologous stem-cell transplantation to the standard regimen of rituximab, cyclophosphamide, doxorubicin, vincristine, and prednisone (R-CHOP). The results show that high plasma PD-L1 level implies poorer prognosis whatever the treatment, more noticeably for patients treated by the standard R-CHOP regimen.

The present invention relates to a method for predicting the survival time of a patient suffering from a diffuse large B-cell lymphoma (DLBCL) comprising the step of i) determining the level of sPD-L1 in a blood sample obtained from the patient ii) comparing the level determined at step i) with a predetermined reference value and iii) concluding that the patient has a poor prognosis when the level determined at step i) is higher than the predetermined reference value or concluding that the patient has a good prognosis when the level determined at step i) is lower than the predetermined reference value.

As used herein the term “PD-1” has its general meaning in the art and refers to the programmed death-1 receptor. PD-1 is a type I transmembrane protein, belonging to the CD28-B7 signalling family of receptors that includes CD28, cytotoxic T-lymphocyte-associated antigen 4 (CTLA-4), inducible costimulator (ICOS), and B- and T-lymphocyte attenuator (BTLA) (Greenwald R J et al., 2005, Riley J L et aL., 2005). So far, two ligands of PD-1 have been identified: PD-L1 (B7-H1) and PD-L2 (B7-DC). Dysfunction of the PD1/PD-L1 pathway is involved in the pathogenesis of various immunological disorders including autoimmunity (Okazaki T et al., 2005), and immunodeficient conditions associated with chronic viral infections (Barber D L et al., 2006, Freeman G J et al., 2006).

As used herein the term “PD-L1” has its general meaning in the art and refers to the programmed death-1 receptor ligand 1. PD-L1 has been characterized as type I transmembrane proteins triggering the PD1 inhibiting effect (Keir M E et al., 2005).

As use herein the term “sPD-L1” has its general meaning in the art and refers to the soluble form of PD-L1. The soluble form of PD-L1 has been described in renal cell carcinoma (Frigola X, Inman B A, Lohse C M, Krco C J, Cheville J C, Thompson R H, Leibovich B, Blute M L, Dong H, Kwon E D. Identification of a soluble form of B7-H1 that retains immunosuppressive activity and is associated with aggressive renal cell carcinoma. Clin Cancer Res. 2011 Apr. 1; 17(7):1915-23. doi: 10.1158/1078-0432.CCR-10-0250. Epub 2011 Feb. 25.)

By “blood sample” is meant a volume of whole blood or fraction thereof, e.g., serum, plasma, etc. The blood sample may be prepared according to any well known method in the art. Typically, a plasma sample (either BD™ P100, EDTA, citrate, or heparin plasma sample) collected from a single venipuncture, or a pooled serum or plasma sample may be prepared.

The predetermined reference value may be determined by any well known method in the art.

Typically, the predetermined reference value may be determined by carrying out a method comprising the steps of

a) providing a collection of blood samples from diffuse large B-cell lymphoma (DLBCL) patients;

b) providing, for each blood sample provided at step a), information relating to the actual clinical outcome for the corresponding diffuse large B-cell lymphoma (DLBCL) patient (i.e. the duration of the overall survival (OS));

c) providing a serial of arbitrary quantification values;

d) determining the level of sPD-L1 for each blood sample contained in the collection provided at step a);

e) classifying said blood samples in two groups for one specific arbitrary quantification value provided at step c), respectively: (i) a first group comprising blood samples that exhibit a quantification value for level that is lower than the said arbitrary quantification value contained in the said serial of quantification values; (ii) a second group comprising blood samples that exhibit a quantification value for said level that is higher than the said arbitrary quantification value contained in the said serial of quantification values; whereby two groups of blood samples are obtained for the said specific quantification value, wherein the blood samples of each group are separately enumerated;

f) calculating the statistical significance between (i) the quantification value obtained at step e) and (ii) the actual clinical outcome of the patients from which blood samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the level of sPD-L1 has been assessed for 100 blood samples of 100 patients. The 100 samples are ranked according to the level of sPD-L1. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, for each of the 99 groups of two subsets, estimates of survival were calculated using the Kaplan Meier method (or any other survival analysis method) and tested with the log-rank test (or any other suitable test). The predetermined reference value is then selected such as the discrimination based on the criterion of the minimum p value is the strongest. In other terms, the level of sPD-L1 corresponding to the boundary between both subsets for which the p value is minimum is considered as the predetermined reference value. It should be noted that the predetermined reference value is not necessarily the median value of levels of sPD-L1.

The setting of a single “cut-off” value thus allows discrimination between a poor and a good prognosis with respect to OS for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” (for which the p value is the lowest) value as described above. For example, on a hypothetical scale of 1 to 10, if the ideal cut-off value (the value with the highest statistical significance) is 5, a suitable (exemplary) range may be from 4-6. Therefore, a patient may be assessed by comparing values obtained by measuring the level of sPD-L1, where values greater than 5 reveal a poor prognosis and values less than 5 reveal a good prognosis. In a another embodiment, a patient may be assessed by comparing values obtained by measuring the level of sPD-L1 and comparing the values on a scale, where values above the range of 4-6 indicate a poor prognosis and values below the range of 4-6 indicate a good prognosis, with values falling within the range of 4-6 indicating an intermediate prognosis.

Typically, the predetermined reference value may be 1.524 ng/ml as described in EXAMPLE 1.

According to the invention, the measure of the level of sPD-L1 can be performed by a variety of techniques. Typically, the methods may comprise contacting the sample with a binding partner capable of selectively interacting with sPD-L1 in the sample. In some aspects, the binding partners are antibodies, such as, for example, monoclonal antibodies or even aptamers as above described.

The aforementioned assays generally involve the binding of the partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The level of sPD-L1 may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

An exemplary biochemical test for identifying specific proteins employs a standardized test format, such as ELISA test, although the information provided herein may apply to the development of other biochemical or diagnostic tests and is not limited to the development of an ELISA test. It is understood that commercial assay enzyme-linked immunosorbant assay (ELISA) kits for various plasma constituents are available. Therefore ELISA method can be used, wherein the wells of a microtiter plate are coated with a set of antibodies which recognize sPD-L1. A sample containing or suspected of containing sPD-L1 is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Measuring the level of sPD-L1 (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e. g., retention time, for that compound and measured using standard techniques.

Alternatively, the separated compounds may be detected and measured by, for example, a mass spectrometer. Typically, levels of immunoreactive sPD-L1 in a sample may be measured by an immunometric assay on the basis of a double-antibody “sandwich” technique, with a monoclonal antibody specific for sPD-L1. According to said embodiment, said means for measuring sPD-L1 level are for example i) a sPD-L1 buffer, ii) a monoclonal antibody that interacts specifically with sPD-L1, iii) an enzyme-conjugated antibody specific for sPD-L1 and a predetermined reference value of sPD-L1. Such a method is for example described in Chen Y, Wang Q, Shi B, Xu P, Hu Z, Bai L, Zhang X. Development of a sandwich ELISA for evaluating soluble sPD-L1 (CD274) in human sera of different ages as well as supernatants of sPD-L1+ cell lines. Cytokine. 2011 November; 56(2):231-8. doi: 10.1016/j.cyto.2011.06.004. Epub 2011 Jul. 5.

In a particular embodiment, the method of the invention is performed at the time of diagnosis.

In a particular embodiment, the patient has received a treatment of its lymphoma, typically a R-CHOP treatment. In this case the method of the invention will be particularly suitable for determining whether the patient will respond well or not to the treatment (e.g. a R-CHOP treatment).

Accordingly, the present invention also relates to a method for determining whether a patient suffering from a DLBCL will respond well or not to a treatment (e.g. a R-CHOP treatment) comprising the step of i) determining the level of sPD-L1 in a blood sample obtained from the patient ii) comparing the level determined at step i) with a predetermined reference value and iii) concluding that the patient will respond well to the treatment when the level determined at step i) is lower than the predetermined reference value or concluding that the patient will not respond well to the treatment when the level determined at step i) is higher than the predetermined reference value.

The term “R-CHOP” has its general meaning in the art and refers to a Rituximab combination therapy that includes a CHOP regimen of cyclophosphamide, doxorubicine, vincristine, and prednisone.

A further object of the invention relates to a kit for performing the above described method, said kit comprising means for measuring the level of sPD-L1 in the blood sample obtained from the patient.

In a particular embodiment, said means for measuring the level of sPD-L1 is an antibody that interacts specifically with sPD-L1. In another embodiment, said means for measuring the level of sPD-L1 may be an aptamer or any other binding partner that specifically recognizes sPD-L1.

Said binding partner can be tagged for an easier detection. It may or may not be immobilized on a substrate surface (e.g., beads, array, and the like). For example, the kit may include an array for predicting the risk of having a cardiovascular event as provided herein. Alternatively, a substrate surface (e.g. membrane) may be included in an inventive kit for immobilization of the binding partner (e.g., via gel electrophoresis and transfer to membrane).

In addition, a kit of the invention generally also comprises at least one reagent for the detection of a complex between binding partner included in the kit and biomarker of the invention.

Depending on the procedure, the kit may further comprise one or more of: extraction buffer and/or reagents, western blotting buffer and/or reagents, and detection means. Protocols for using these buffers and reagents for performing different steps of the procedure may be included in the kit.

The different reagents included in a kit of the invention may be supplied in a solid (e.g. lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit comprises instructions for using its components for the prediction of a cardiovascular event in a patient according to a method of the invention. Instructions for using the kit according to methods of the invention may comprise instructions for processing the biological sample obtained from the patient and/or for performing the test, or instructions for interpreting the results. A kit may also contain a notice in the form prescribed by a governmental agency regulating the manufacture, use or sale of pharmaceuticals or biological products.

A further object relates to a method for the treatment of a diffuse large B-cell lymphoma (DLBCL) in a patient in need thereof comprising the step consisting of i) predicting the survival time of the patient according to the method as above described and ii) administering the patient with a therapeutically amount of a PD-1 antagonist when it is concluded that the patient has a poor prognosis at step i).

In a particular embodiment, the patient was previously administered with a R-CHOP treatment.

The term “PD-1 antagonist” means any molecule that attenuates inhibitory signal transduction mediated by the binding of PD-L1 to PD-1. In specific examples of the invention, a PD-1 antagonist is a molecule that inhibits, reduces, abolishes or otherwise reduces inhibitory signal transduction through the PD-1 receptor signalling pathway. Such decrease may result where: (i) the PD-1 antagonist of the invention binds to a PD-1 receptor without triggering signal transduction, to reduce or block inhibitory signal transduction mediated by PD-L1; (ii) the PD-1 antagonist binds to PD-L1, preventing its binding to PD-1; (iii) the PD-1 antagonist binds to, or otherwise inhibits the activity of, a molecule that is part of a regulatory chain that, when not inhibited, has the result of stimulating or otherwise facilitating PD-1 inhibitory signal transduction mediated by PD-L1; or (iv) the PD-1 antagonist inhibits PD-1 expression or PD-L1 expression, especially by reducing or abolishing expression of one or more genes encoding PD-1 or one PD-L1.

In a particular embodiment the PD-1 antagonist is an antibody selected from the group consisting of anti-PD-1 antibodies or anti-PD-L1 antibodies. Examples of anti-PD-1 antibodies include, but are not limited to, those described in the following publications: PCT/IL03/00425 (Hardy et al., WO/2003/099196) PCT/JP2006/309606 (Korman et al., WO/2006/121168) PCT/US2008/008925 (Li et al., WO/2009/014708) PCT/JP03/08420 (Honjo et al., WO/2004/004771) PCT/JP04/00549 (Honjo et al., WO/2004/072286) PCT/IB2003/006304 (Collins et al., WO/2004/056875) PCT/US2007/088851 (Ahmed et al., WO/2008/083174) PCT/US2006/026046 (Korman et al., WO/2007/005874) PCT/US2008/084923 (Terrett et al., WO/2009/073533), Berger et al., Clin. Cancer Res., Vol. 14, pp. 30443051 (2008). A specific example of an anti-PD-1 antibody is MDX-1 106 (see Kosak, US 20070166281 (pub. 19 Jul. 2007) at par. 42). Examples of anti-PD-L1 antibodies include, but are not limited to, those described in the following publications: PCT/US06/022423 (WO/2006/133396, pub. 14 Dec. 2006) PCT/US07/088,851 (WO/2008/083174, pub. 10 Jul. 2008) US 2006/0110383 (pub. 25 May 2006). A specific example of an anti-PD-L1 antibody useful in the methods of the invention is MDX-1105 (WO/2007/005874, published 11 Jan. 2007) which is a high-affinity, fully human, PD-L1-specific, IgG4 (S228P) monoclonal antibody that inhibits the binding of PD-L1 to both PD-1.

By a “therapeutically effective amount” of the PD-1 antagonist as above described is meant a sufficient amount of the PD-1 antagonist to treat DLBCL. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PD-1 antagonist of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The PD-1 antagonist of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The PD-1 antagonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1 shows the diagram of the whole cohort.

FIG. 2. OS curve according to the assigned arm at randomization (top) and according to the low-level and high-level of PD-L1 (bottom).

FIG. 3A. Overall survival curves for the GOELAMS 075 cohort. Intention-to-treat overall survival curves according to PD-L1 levels for patients assigned to the R-CHOP arm (top) and the high-dose chemiotherapy arm (bottom).

FIG. 3B. Per-protocol overall survival curves according to PD-L1 levels for the R-CHOP (top), the high-dose chimiotherapy (middle) and salvage therapy (bottom) populations.

FIG. 4A. Comparison of soluble PD-L1 levels between GOELAMS 075 patients at diagnosis and healthy controls.

FIG. 4B. Comparison of soluble PD-L1 levels between GOELAMS 075 patients at diagnosis and after complete remission and healthy controls.

### EXAMPLE

### Elevated Plasma PD-L1 Protien Level in Aggressive Diffuse Large B-Cell Lymphoma Impacts Patients' Outcome

Methods:

GOELAMS 075 Patients

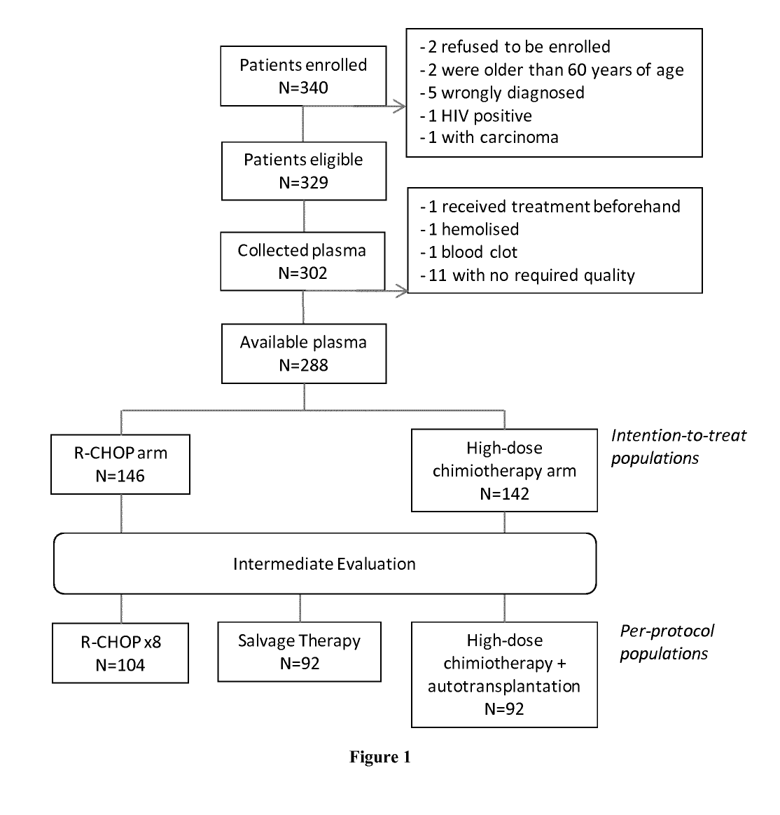

The Groupe Ouest-Est des Leucémies et des Autres Maladies du Sang (GOELAMS) 075 study is a multicentric randomized trial designed to translate in the rituximab era previous data showing the superiority of high-dose chemotherapy with autologous stem-cell support compared to CHOP in adults with disseminated aggressive lymphoma⁹. To this end, at the time of diagnosis, adults with untreated confirmed diffuse large B-cell lymphoma were randomly assigned to receive eight courses of CHOP plus rituximab (R-CHOP) every 14 days or high-dose chemotherapy associated to rituximab before autologous stem cell support (clinicaltrials.gov: NCT00561379). Patients were required to have a Ann Arbor stage of III or IV, or I or II with bulk greater or equal to 7 cm. The trial was approved by the ethics committee of the Centre Hospitalier Universitaire de Nantes, Nantes, France, and all patients gave written informed consent. Between January 2005 and June 2010, 16 centers participating in GOELAMS enrolled 340 consecutive patients from 18 to 60 years old with histologically proved CD20+ diffuse large B-cell lymphoma according the World Health Organization (WHO) classification introduced in 2001 and completed in 2008 (FIG. 1). Randomization was performed according to center, with no further stratification. Eleven patients were found to be ineligible. Eleven patients were found to be ineligible for the trial. Of the 329 patients left, plasma was not collected for 27 patients and not eligible for an additional 14 patients. Of the remaining 288 patients with available plasma, 146 patients were assigned to the R-CHOP arm and 142 to the high-dose chemotherapy arm (intention-to-treat populations). An intermediate evaluation of response was assessed after the first 4 courses of R-CHOP or the 3 courses of high-dose chemotherapy associated to rituximab administrated before autologous stem cell transplantation, by means of standard CT scan and positron-emission tomographic (PET) scan. As a general rule, patients achieving a negative PET would, depending on their intended treatment, undergo four additional cycles of R-CHOP or autotransplantation, while to the other patients were proposed a salvage therapy which could vary among the centers and that was related to a previous scheme that included 3 courses of dexamathasone, cisplatin, and cytarabine (DHAP) plus rituximab before autologous stem cell transplantation. Per-protocol populations included 104 patients in the R-CHOP arm, 92 patients in the high-dose chemotherapy with autotransplantation and 92 patients in the salvage therapy.

Data and Tissue Collections

Plasma was obtained from 288 GOELAMS patients at diagnosis, from 75 GOELAMS patients who reached complete remission six months after the end of their treatment and from 100 healthy volunteers from the French National Blood Service matched for age and sex with the first 200 enrolled patients. All the plasma samples were collected into anti-proteasic P100® tubes (BD™), shipped at room temperature within 24 hours to the Rennes core lab and stored as 500 μl aliquots at −80° upon reception. Clinical data were obtained at diagnosis, intermediate, final and follow-up evaluations. Tissue and data collections were approved by the National Ethics Committee.

Soluble PD-L1 Measurement

Soluble PD-L1 was measured using an enzyme-linked immunosorbent assay (USCNK PDCD1LG1 ELISA kit), according to the manufacturer instructions. The dosages were performed at the University Hospital of Rennes. The minimum detectable dose of PDLL was less than 0.039 ng/mL and minimum quantitative range was 0.156 ng/mL. Each sample was analyzed in duplicates; the intra-assay and inter-assay coefficients of variation were <20%. The same assay and procedure were used to measure PD-L1 levels for all studied cohorts.

Statistical Analysis

Patients' characteristics were described by their median and range [min:max] for quantitative data, absolute and relative frequencies for qualitative data. Differences of characteristics between groups were calculated with the Mann-Whitney U test for quantitative data and the Chi2 test for qualitative data.

Overall survival was defined as the time from randomization to either the last follow up or death from any cause. Estimates of survival was calculated according to the Kaplan-Meier method and tested with the log-rank test. Differences between the results of comparative tests were considered significant if the two-sided p-value was less than 0.05. Confidence interval was stated at the 95th confidence level.

The PD-L1 prognostic threshold was determined with the R MaxStat function on the 288 plasma collected at diagnosis for the GOELAMS 075 trial¹¹. Associations of soluble PD-L1 as a binary variable with various clinical factors were evaluated by means of the Chi2 test for quantitative data and the Mann-Witney U test for quantitative data.

The prognostic value of binary PD-L1 was assessed on the GOELAMS 075 patients, first on the entire cohort, then on the intention-to-treat populations.

All variables statistically significant with a p-value less than 10% in univariate Cox analyses were entered in the multivariate Cox proportional hazards model to assess the independent effect of prognostic variables on the outcome using the backward elimination procedure.

Soluble PD-L1 was further analyzed as a continuous variable. The ability of plasma PD-L1 levels to distinguish patients from controls was evaluated by means of the Mann-Whitney test for independent samples on the 288 patients and a subset of 60 healthy volunteers. The ability of plasma PD-L1 levels to distinguish patients with complete remission from patients at diagnosis was evaluated by means of the Wilcoxon-matched test for paired samples on a subset of 12 patients. Statistical analyses were performed with the use of GraphPad Prism (version 5.01, GraphPad Software Inc., La Jolla, Calif.) and the R software (version 2.14) (R Development Core Team (2011). R: A language and environment for statistical computing. R Foundation for Statistical Computing, Vienna, Austria. http://www.R-project.org).

Results:

A Cohort of 288 Patients With Available Plasma:

The 288 patients with available plasma demonstrated no statistical difference with the cohort of 329 patients eligible to the GOELAMS 075 trial (data not shown). Patients' demographic, baseline disease characteristics and responses at the intermediate evaluation are listed in Table 1. Overall 50 patients died, 26 (17.8%) patients in the R-CHOP intended treatment, 24 (16.8%) in the high-dose chemotherapy intended treatment. Seven patients (2.4%) stopped their treatment due to toxicity, two of them consequently died. Of the 288 patients, there were 107 (37.2%) complete remissions and 69 (24%) unconfirmed complete remissions. Median follow-up was of 41.45 months, 42 patients experienced progression, death or relapse as first event. Three-year overall survival was estimated at 84.8% (CI: 80.5-89). There was no statistically significant difference between the R-CHOP and the high-dose chemotherapy arms (p=0.97).

High Plasma PD-L1 Level Impacts Patient's Outcome:

The optimal threshold for soluble PD-L1 measured at diagnosis was equal to 1.524 ng/mL. Eighty-eight (30.5%) patients had a high level of soluble PD-L1 (>1.524 ng/mL). The numbers of patients in the assigned arms at randomization were strictly comparable in the two PD-L1 groups (p=0.38).

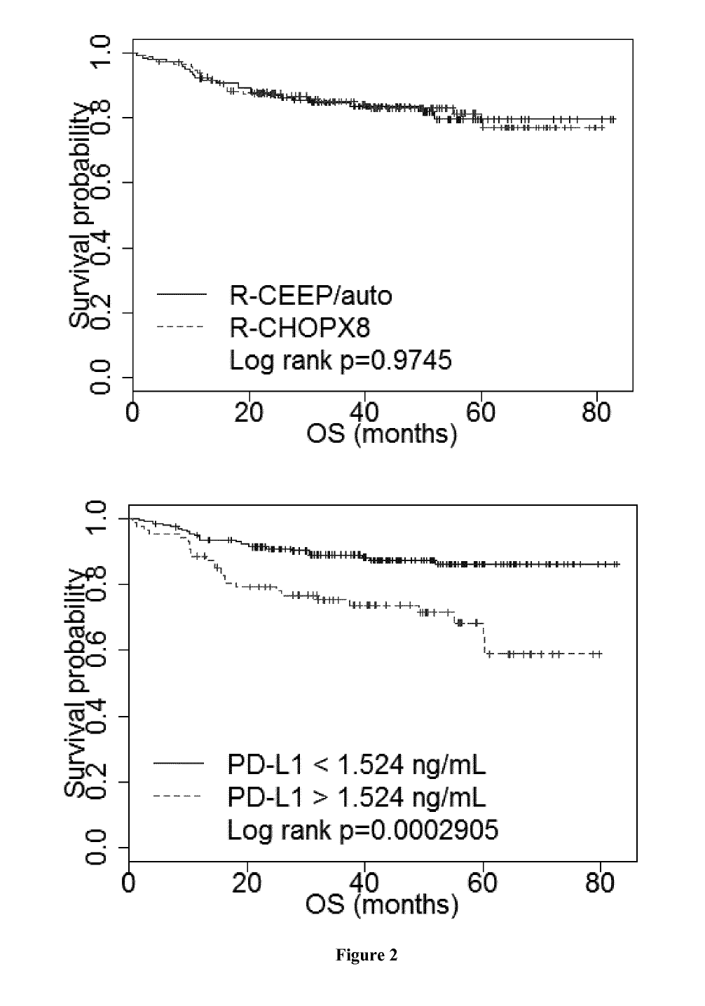

The high-level PD-L1 group was associated with poorer prognosis (p=0.0002905) with a 3-year overall survival estimated at 75.3% (CI: 66.6-85.1) (respectively 89% (CI: 84.6-93.5) for the low-level PD-L1 group), while there was no statistical difference in overall survival between the two assigned arms at randomization (FIG. 2).

The high-level PD-L1 group was significantly associated with bone marrow involvement, more than one extranodal localizations, 2-4 performance status, abnormal level of β2microglobulin, abnormal absolute lymphocyte count (suppl. table 1).

The univariate analysis identified older age and abnormal level of β2microglobulin as factors associated with poor prognostic, as well as high age-adjusted IPI score, bone marrow involvement, abnormal lymphocyte-monocyte score and high level of soluble PD-L1 (Suppl. Table 2). The multivariate Cox method identified three factors with p<5% (Table 2).

High Plasma PD-L1 Level Implies Poorer Prognosis for R-CHOP-Treated Patients:

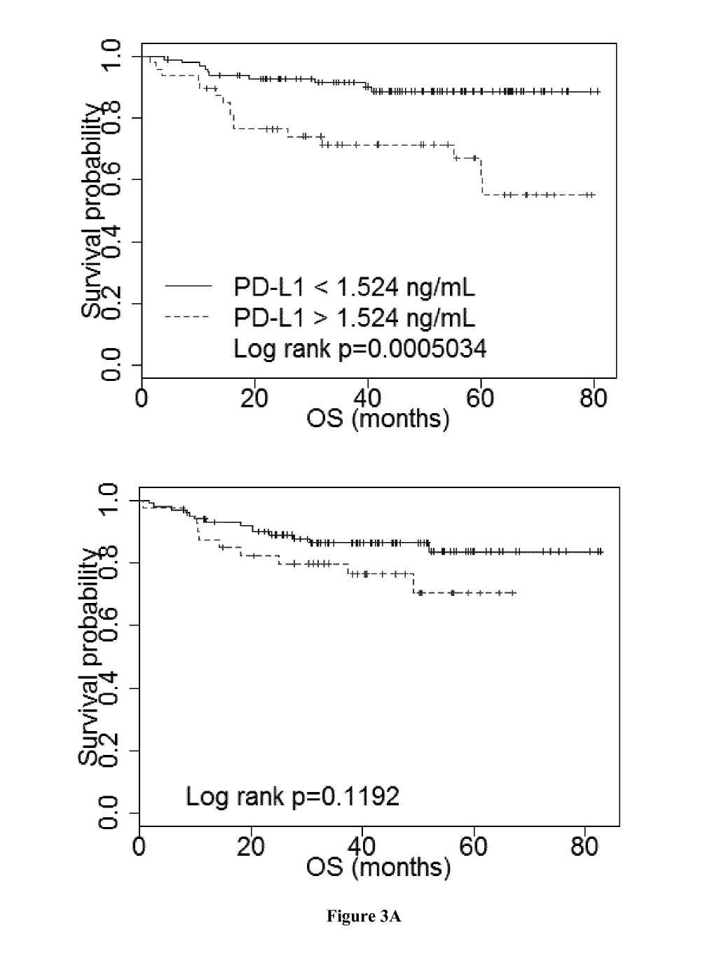

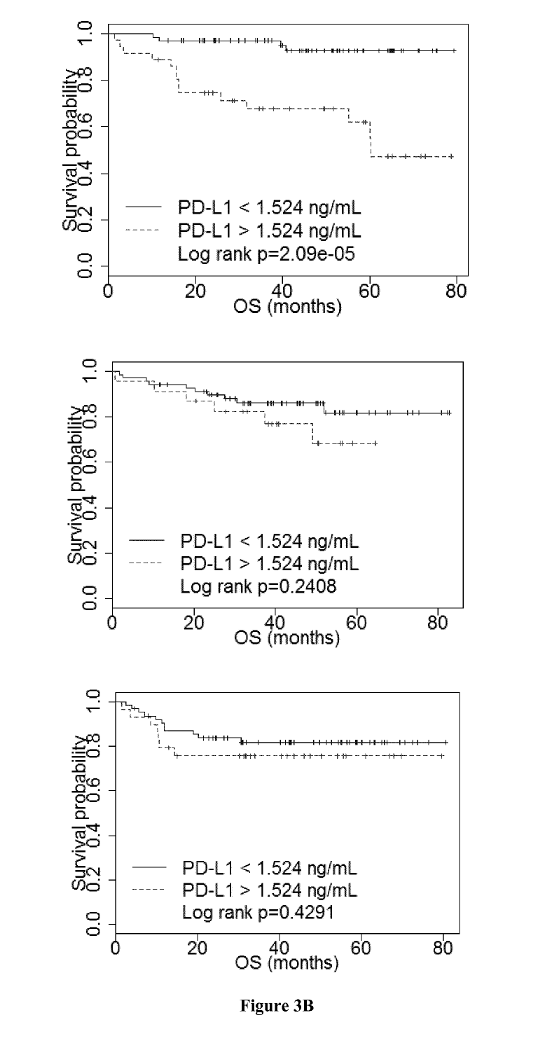

Intention-to-treat analysis showed that the high-level PD-L1 group for the 146 patients randomized in the R-CHOP arm was associated with poorer prognosis (p=0.000503,FIG. 3A top) with a 3-year overall survival estimated at 71.4% (CI:59.4-86) than the low-level PD-L1 group (91.5% (CI:86.1-97.3) 3-year overall survival). Plasma PD-L1 did not show a statistical difference for the 142 patients randomized in the high-dose chimiotherapy arm (p=0.119, FIG. 3A bottom) between the high and low level groups (3-year overall survival respectivally of 79.8% (CI: 68.2-93.3) and 86.5% (CI:79.9-93.6)).

The 104 patients of the R-CHOP per-protocol population demonstrated poorer prognosis (p=2.09 10-5, FIG. 3B top) for high levels of PD-L1 measured at time of diagnosis (67.7% (CI:53.6-85.6) 3 year overall survival) than for low levels (97.1% (CI: 93.1-100) 3-year overall survival). The 92 patients of the high-dose chimiotherapy per-protocol population demonstrated no statistical difference (p=0.24, FIG. 3B middle) between high PD-L1 levels (82.4% (CI:68.1-99.7) 3-year overall survival) and low PD-L1 levels (86.1% (CI:78-95) 3 year overall survival). The 92 patients having received salvage therapy demonstrated no statistical difference (p=0.43, FIG. 3B bottom) between high PD-L1 levels (75.7% (CI:61.5-93.1) 3-year overall survival) and low PD-L1 levels (81.8% (CI:72.5-92.2) 3 year overall survival).

Characterization of Plasma PD-L1 Levels in DLBCL Patients

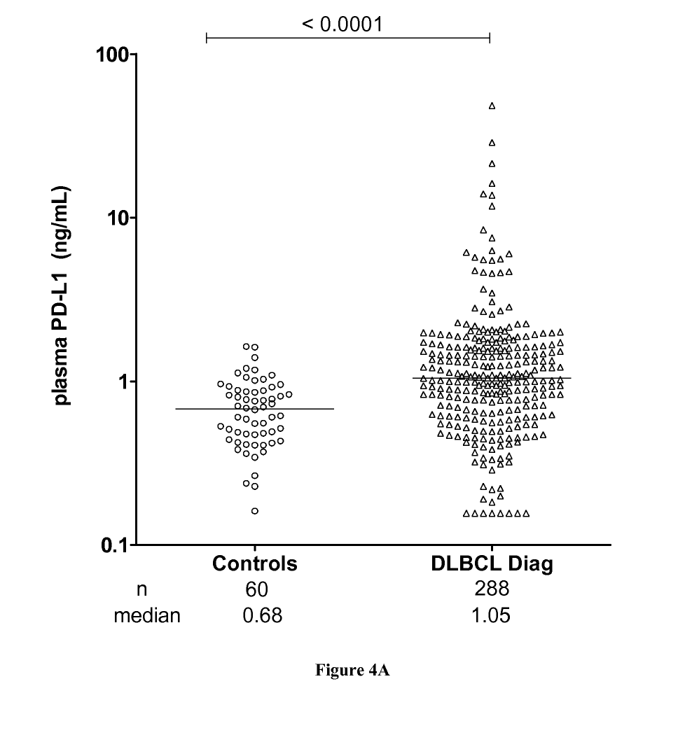

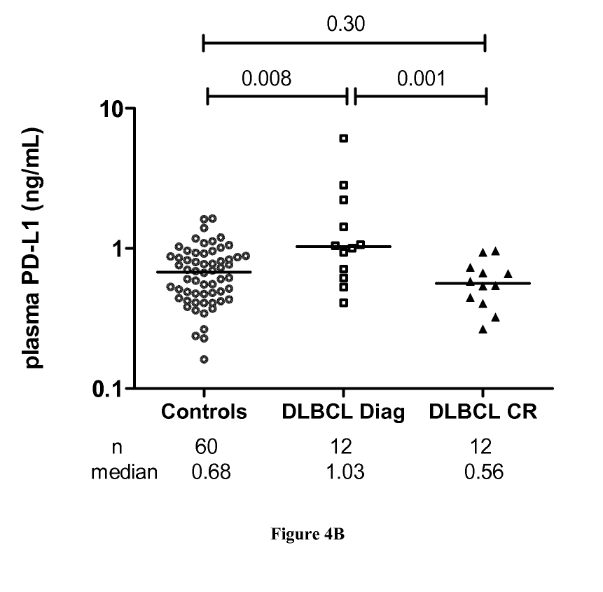

Circulating levels of plasma PD-L1 of the 288 newly diagnosed patients with aggressive DLBCL (median: 1.05 ng/mL, mean: 1.84±3.89 ng/mL, range: 0.15-48.44 ng/mL) were significantly increased compared to the 60 healthy subjects (median: 0.68 ng/mL, mean: 0.70±0.32 ng/mL, range: 0.16-1.63 ng/mL) (p<0.0001, FIG. 4A). While 30.6% of patients had PD-L1 levels greater than 1.524 ng/mL, 58 (96.7%) controls had a level lower than the threshold.

Plasma PD-L1 level as a quantitative variable was found significantly higher for patients with bone marrow involvement (p=0.0093), abnormal β2microglobulin (p=0.0085), more than one extranodal localization (p=0.0492) or 2-3 aa-IPI scores (p=0.0508). It was not significantly different for any of the other patients' characteristics, including positive vs negative TEP response at the intermediate evaluation (data not shown).

Plasma PD-L1 levels of 12 patients with complete remission and off therapy for 6 months (median: 0.56 ng/mL, mean: 0.59±0.22 ng/mL, range: 0.27-0.96 ng/mL) were significantly decreased compared to their levels at diagnosis (median: 1.03 ng/mL, mean: 1.58±1.60 ng/mL, range: 0.41-6.1 ng/mL) (p<0.001, FIG. 4B). The PD-L1 levels for all 12 patients had decreased under the threshold after complete remission. No statistical difference was found between the plasma PD-L1 levels of the 12 patients in complete remission and the 60 healthy controls (p=0.30).

TABLE 1
Characteristics of Patients with aggressive diffuse large B-cell at
Diagnosis and at the Intermediate Evaluation (n = 288).

| Characteristic | N (%) |
|---|---|
| At Diagnosis | |
| Age - median yr [range] | 49.5 [18-60] |
| Sex | |
| Male | 169 (59) |
| Female | 119 (41) |
| Performance Status | |
| 0-1 | 244 (85) |
| 2-4 | 44 (15) |
| Ann Arbor Stage after TEP | |
| I-II | 67 (23) |
| III-IV | 216 (75) |
| Symptoms | |
| A | 165 (57) |
| B | 122 (42) |
| LDH | |
| Normal | 74 (26) |
| Elevated | 213 (74) |
| Extranodal Localization | |
| <2 | 155 (54) |
| ≧2 | 118 (41) |
| Bulky Disease | |
| <7 cm | 106 (37) |
| ≧7 cm | 175 (61) |
| Bone Marrow Involvement | |
| No | 220 (76) |
| Yes | 46 (16) |
| aa-IPI Score | |
| 0-1 | 116 (40) |
| 2-3 | 172 (60) |
| At the Intermediate Evaluation | |
| CT Scan Response | |
| Complete Remission | 107 (37) |
| Unconfirmed Complete Remission | 69 (24) |
| Partial Remission >50% | 88 (31) |
| Stable Disease | 14 (5) |
| Progression | 6 (2) |
| Death | 2 (1) |
| TEP Response | |
| Negative | 174 (60) |
| Positive | 101 (35) |

TABLE 2
Hazard Ratios for the association between various
factors and the overall survival.

| Variable | Hazard Ratio (95% CI) | P Value |
|---|---|---|
| PD-L1 | | |
| <1.524 ng/mL (Low Level) | Reference | |
| ≧1.524 ng/mL (High Level) | 1.94 (1.04-3.62) | 0.037 |
| Bone Marrow Involvement | | |
| No | Reference | |
| Yes | 2.55 (1.34-4.87) | 0.0045 |
| lymphocyte-Monocyte Score | | |
| Normal | Reference | |
| Abnormal | 4.75 (1.14-19.78) | 0.0321 |
| aa-IPI Score | | |
| 0-1 | Reference | |
| 2-3 | 1.52 (0.74-3.089) | 0.252 |

SUPPLEMENTAL TABLE 1
Patients' characteristics according to soluble PD-L1 measures.

| | Binary PD-L1 | | |
|---|---|---|---|
| | Low level | High level | |
| | PD-L1 | PD-L1 | |
| | (N) | (N) | P Value |
| Bone marrow involvement | | | 0.00073 |
| No | 161 | 59 | |
| Yes | 22 | 24 | |
| β2microglobulin | | | 0.0021 |
| Normal | 94 | 22 | |
| Abnormal | 63 | 35 | |
| Extranodal localization | | | 0.0019 |
| <2 | 119 | 36 | |
| ≧2 | 70 | 48 | |
| aa-IPI Score | | | 0.0928 |
| 0-1 | 87 | 29 | |
| 2-3 | 113 | 59 | |
| Intention-to-treat | | | 0.38 |
| R-CEEP-auto | 102 | 40 | |
| R-CHOP | 98 | 48 | |
| Sex | | | 0.92 |
| Male | 117 | 52 | |
| Female | 83 | 36 | |
| Age | 49 y.o. | 50.7 y.o. | 0.32 |
| Performance status | | | 0.0192 |
| 0-1 | 176 | 68 | |
| 2-4 | 24 | 20 | |
| Ann Arbor stage after TEP | | | 0.276 |
| I-II | 50 | 17 | |
| III-IV | 146 | 70 | |
| LDH | | | 0.621 |
| Normal | 53 | 21 | |
| Elevated | 146 | 67 | |
| Symptoms | | | 0.118 |
| A | 121 | 44 | |
| B | 79 | 43 | |
| Bulky disease | | | 0.0557 |
| <7 cm | 68 | 38 | |
| ≧7 cm | 113 | 44 | |
| Lymphocyte counts | 1.1 | 0.925 | 0.034 |
| Monocyte counts | 0.75 | 0.745 | 0.297 |
| CT-Scan response | | | 0.681 |
| Complete remission | 76 | 31 | |
| No complete remission | 123 | 56 | |
| TEP response | | | 0.837 |
| Negative | 122 | 52 | |
| Positive | 72 | 29 | |

SUPPLEMENTAL TABLE 2
Overall survival according to patients' characteristics.

| | Overall Survival | | |
|---|---|---|---|
| | (Kaplan-Meier) | | |
| | N | death | P Value |
| Intention-to-treat | | | 0.9745 |
| R-CEEP-auto | 142 | 24 | |
| R-CHOP | 146 | 26 | |
| Sex | | | 0.982 |
| Male | 169 | 29 | |
| Female | 119 | 21 | |
| Age | | | 0.0005445 |
| <54 y.o. | 191 | 22 | |
| ≧54 y.o. | 97 | 28 | |
| Performance status | | | 0.103 |
| 0-1 | 244 | 39 | |
| 2-4 | 44 | 11 | |
| Bone marrow involvement | | | 4.025 10-5 |
| No | 220 | 28 | |
| Yes | 46 | 18 | |
| β2microglobulin | | | 0.05575 |
| Normal | 115 | 12 | |
| Abnormal | 91 | 18 | |
| Extranodal localization | | | 0.000147 |
| <2 | 155 | 14 | |
| ≧2 | 118 | 32 | |
| aa-IPI Score | | | 0.02294 |
| 0-1 | 116 | 13 | |
| 2-3 | 172 | 37 | |
| Ann Arbor stage after TEP | | | 0.04336 |
| I-II | 67 | 6 | |
| III-IV | 216 | 44 | |
| LDH | | | 0.278 |
| Normal | 74 | 10 | |
| Elevated | 213 | 40 | |
| Symptoms | | | 0.18 |
| A | 165 | 24 | |
| B | 122 | 25 | |
| Bulky disease | | | 0.115 |
| <7 cm | 106 | 23 | |
| ≧7 cm | 175 | 23 | |
| Lymphocyte-Monocyte Score | | | 0.02235 |
| Normal (Ly>1.1 and Mo<0.76) | 58 | 4 | |
| Abnormal | 219 | 44 | |
| Soluble PD-L1 | | | 0.0002905 |
| <1.524 ng/mL | 200 | 24 | |
| ≧1.524 ng/mL | 88 | 26 | |

### REFERENCES

Throughout this application, various references describe the state of the art to which this invention pertains. The disclosures of these references are hereby incorporated by reference into the present disclosure.

1. Lenz G, Staudt L M. Aggressive lymphomas. N Engl J Med; 362:1417-29.

2. Chambers C A, Kuhns M S, Egen J G, Allison J P. CTLA-4-mediated inhibition in regulation of T cell responses: mechanisms and manipulation in tumor immunotherapy. Annu Rev Immuno12001; 19:565-94.

3. Waterhouse P, Penninger J M, Timms E, et al. Lymphoproliferative disorders with early lethality in mice deficient in Ctla-4. Science 1995; 270:985-8.

4. Brahmer J R, Tykodi S S, Chow L Q, et al. Safety and activity of anti-PD-L1 antibody in patients with advanced cancer. N Engl J Med; 366:2455-65.

5. Hodi F S, O'Day S J, McDermott D F, et al. Improved survival with ipilimumab in patients with metastatic melanoma. N Engl J Med; 363:711-23.

6. Topalian S L, Hodi F S, Brahmer J R, et al. Safety, activity, and immune correlates of anti-PD-1 antibody in cancer. N Engl J Med; 366:2443-54.

7. Rosenwald A, Wright G, Leroy K, et al. Molecular diagnosis of primary mediastinal B cell lymphoma identifies a clinically favorable subgroup of diffuse large B cell lymphoma related to Hodgkin lymphoma. J Exp Med 2003; 198:851-62.

8. Xerri L, Chetaille B, Serriari N, et al. Programmed death 1 is a marker of angioimmunoblastic T-cell lymphoma and B-cell small lymphocytic lymphoma/chronic lymphocytic leukemia. Hum Pathol 2008; 39:1050-8.

9. Milpied N, Deconinck E, Gaillard F, et al. Initial treatment of aggressive lymphoma with high-dose chemotherapy and autologous stem-cell support. N Engl J Med 2004; 350:1287-95.

10. Philip T, Guglielmi C, Hagenbeek A, et al. Autologous bone marrow transplantation as compared with salvage chemotherapy in relapses of chemotherapy-sensitive non-Hodgkin's lymphoma. N Engl J Med 1995; 333:1540-5.

11. Hothorn T., Lausen B. On the exact distribution of maximally selected rank statistics. Computational Statistics & Data Analysis, 2003; 43:121-137.

1. A method for predicting the survival time of a patient suffering from a diffuse large B-cell lymphoma (DLBCL) comprising the step of i) determining the level of sPD-L1 in a blood sample obtained from the patient ii) comparing the level determined at step i) with a predetermined reference value and iii) concluding that the patient has a poor prognosis when the level determined at step i) is higher than the predetermined reference value or concluding that the patient has a good prognosis when the level determined at step i) is lower than the predetermined reference value.

2. The method according to claim 1 wherein the patient has received a R-CHOP treatment.

3. A method for determining whether a patient suffering from a DLBCL will respond well or not to a treatment comprising the step of i) determining the level of sPD-L1 in a blood sample obtained from the patient ii) comparing the level determined at step i) with a predetermined reference value and iii) concluding that the patient will respond well to the treatment when the level determined at step i) is lower than the predetermined reference value or concluding that the patient will not respond well to the treatment when the level determined at step i) is higher than the predetermined reference value.

4. A method for the treatment of a diffuse large B-cell lymphoma (DLBCL) in a patient in need thereof comprising steps of i) predicting the survival time of the patient according to the method of claim 1 and ii) administering to the patient a therapeutically effective amount of a PD-1 antagonist when it is concluded that the patient has a poor prognosis at step iii).

5. The method of claim 4 wherein the PD-1 antagonist is an antibody selected from the group consisting of anti-PD-1 antibodies and anti-PD-L1 antibodies.

6. The method of claim 3, wherein said treatment is a R-CHOP treatment.
